(19) United States
(12) Patent Application Publication
(10) Pub. No.: US 2017/0128159 A1
(43) Pub. Date: May 11, 2017

(54) ORTHODONTIC METHOD

(71) Applicant: Akihisa FUKAWA, Shizuoka-shi (JP)

(72) Inventor: Akihisa FUKAWA, Shizuoka-shi (JP)

(21) Appl. No.: 15/007,540

(22) Filed: Jan. 27, 2016

(30) Foreign Application Priority Data
Nov. 11, 2015 (JP) 2015-221095

(51) Int. Cl. A61C 7/00; A61M 5/178; A61C 19/06; A61B 6/03; A61B 6/14

(52) U.S. Cl. CPC A61C 7/002; A61B 6/03; A61B 6/14; A61C 7/008; A61C 19/063; A61M 5/178; A61N 2005/0606

(57) ABSTRACT

An orthodontic method is disclosed wherein images of the oral cavity are taken in advance by means of computed tomography and the images are analyzed. The orthodontic method includes means for causing bone resorption to the alveolar bone and means for applying an orthodontic force to the tooth without using an orthodontic appliance. The means for applying an orthodontic force to the tooth may be sonic wave vibration applied to the tooth. The means for causing bone resorption to the alveolar bone may be sonic wave vibration applied to the alveolar bone, a laser beam applied to the alveolar bone, or a substance involved in bone resorption being injected into a surface of the alveolar bone. The substance involved in bone resorption may be Th17.

### CROSS-REFERENCE TO RELATED U.S. APPLICATIONS

Not applicable.

### STATEMENT REGARDING FEDERALLY SPONSORED RESEARCH OR DEVELOPMENT

Not applicable.

### NAMES OF PARTIES TO A JOINT RESEARCH AGREEMENT

Not applicable.

### REFERENCE TO AN APPENDIX SUBMITTED ON COMPACT DISC

Not applicable.

### BACKGROUND OF THE INVENTION

1. Field of the Invention

The present invention relates to an orthodontic method without using an orthodontic appliance. More particularly, the invention relates to an orthodontic method by taking images of the oral cavity in advance by means of computed tomography and analyzing said images, said orthodontic method comprising means for causing bone resorption to the alveolar bone and means for applying an orthodontic force to the tooth.

2. Description of Related Art Including Information Disclosed Under 37 CFR 1.97 and 37 CFR 1.98.

Japanese Patent Laid-Open Publication No. Hei 8-280711 discloses an orthodontic method by using an orthodontic appliance. It is known that if an orthodontic force is applied to a tooth by an orthodontic appliance, a stress is applied to the periodontal membrane and dental follicle in the direction in which the tooth is to be moved, said stress giving inflammatory stimuli and activating osteoclasts of the alveolar bone, thereby bone resorption being caused.

The center of tooth movement is usually in the tooth root. Since the tooth root is in the alveolar bone, it is not possible to attach an orthodontic appliance to the tooth root. Therefore, a tooth in malposition is physically pulled by means of an orthodontic appliance attached to the coronal portion.

The principle of the orthodontic method is as follows:

A tooth can move within the alveolar bone because the fibrous connective tissue of the dental follicle and the osteoclasts of the periodontal membrane are activated by inflammatory stimuli and the alveolar bone is resorbed. At this time, the tooth receives a load (orthodontic force) in the direction of movement.

In the conventional orthodontic method, the tooth crown is aligned by moving the tooth by applying a force. If an orthodontic force is applied to the tooth, the periodontal membrane on the side opposite to the force is compressed, the alveolar bone being stressed, inflammatory stimuli being generated so as to produce an inflammatory stimulus substance and osteoclasts, then bone resorption being caused by activation. The periodontal membrane on the side to which said force is applied is extended so as to produce osteoblasts, and alveolar bone is formed.

Some dentists perform simulation by computed tomography. However, such simulation is performed for the purpose of explanation for patients, not for the purpose of studying the direction and movement of the tooth by computed tomography in the orthodontic method.

Some people prepare teeth models by a 3D printer on a basis of images of computed tomography and further prepare orthodontic appliances on a basis of said orthodontic appliances.

In the conventional orthodontic method, it is necessary to attach an orthodontic appliance to the tooth crown of the tooth to be moved. The point of action for moving of the tooth is in the tooth crown. The leverage which is the center of movement is usually substantially in the center of the tooth root. This means that the leverage and the point of action are apart from one another. Therefore, the force to be applied to the tooth crown must be larger than the force to be applied to the tooth root. Thus, an excessive force may be applied to the tooth crown.

In the conventional orthodontic method, it is often necessary to fenestrate the gingival and alveolar bone deep into the gingival cheek transition. Such fenestration needs the help of an oral surgeon. Furthermore, there is a risk of the orthodontic appliance attached to the teeth getting out of place. In this case, fenestration may have to be done again. Also, the teeth surrounding tissue may be damaged and cause inflammation which needs dosage.

In the conventional orthodontic method with the orthodontic appliance attached to the teeth, there are further disadvantages as follows: Tooth decay and stomatitis maybe caused. A force applied directly to the tooth may damage the tooth root. The orthodontist has to continue, for a long period of time, cleaning and controlling the teeth and teeth surrounding tissue and adjusting the device for moving the teeth. The patient has foreign-body sensation in the oral cavity. The orthodontic appliance attached to the teeth may harm the appearance. This imposes psychological and physical burden on the patient.

### BRIEF SUMMARY OF THE INVENTION

It is an object of the invention to provide an orthodontic method which does not have the above-mentioned disadvantages of the conventional orthodontic method.

It is another object of the invention to provide an orthodontic method without using an orthodontic appliance.

These and other objects have been attained by the following orthodontic method:

An orthodontic method by taking images of the oral cavity in advance by means of computed tomography and analyzing said images, said orthodontic method comprising means for causing bone resorption to the alveolar bone and means for applying an orthodontic force to the tooth without using an orthodontic appliance.

Said means for applying an orthodontic force to the tooth may be sonic wave vibration applied to said tooth.

Said means for causing bone resorption to the alveolar bone may be sonic wave vibration applied to said alveolar bone.

Said means for causing bone resorption to the alveolar bone may alternatively be a laser beam applied to said alveolar bone.

Said means for causing bone resorption to the alveolar bone may alternatively be a substance involved in bone resorption, said substance being injected into a surface of said alveolar bone.

Said substance involved in bone resorption may be Th17.

In the orthodontic method according to the present invention, images of the oral cavity are taken in advance by means of computed tomography and said images are analyzed. Therefore, the method, distance, etc. of the movement of the tooth suitable for the orthodontics of the patient can be determined three-dimensionally. This point will be described in detail later.

The orthodontic method according to the present invention comprises means for causing bone resorption to the alveolar bone and means for applying an orthodontic force to the tooth. The alveolar bone causes bone resorption and the tooth root is aligned so that the tooth can be moved as desired. Inflammatory stimuli are applied to the alveolar bone on the side to which the tooth is to be moved, osteoclasts being produced and activated so as to cause bone resorption to the alveolar bone. If only said means for causing bone resorption to the alveolar bone is used and said means for applying an orthodontic force to the tooth is not used, only said bone resorption will be finished and the tooth will not move. The tooth will move to the desired position only when both said means for causing bone resorption to the alveolar bone and said means for applying an orthodontic force to the tooth are used.

It is necessary to give a mechanical effect to the portion of the tooth on which a load is put, and to give a thermal effect serving as inflammatory stimuli to the portion causing bone resorption to the alveolar bone.

In a case where said means for applying an orthodontic force to the tooth is sonic wave vibration applied to said tooth, it is possible to apply a force to the tooth from a desired three-dimensional angle. By focusing the sonic wave vibration, it is possible to give effect to a desired portion of the tooth locally in a pinpoint manner.

In a case where said means for causing bone resorption to the alveolar bone is sonic wave vibration applied to said alveolar bone, desirable inflammatory stimuli are given to the alveolar bone, thereby osteoclasts being produced and activated so as to cause bone resorption to the alveolar bone. The sonic wave vibration produces a thermal effect serving as an inflammatory stimulus and a mechanical effect serving as an orthodontic force. By focusing the sonic wave vibration, it is possible to give effect to a desired portion of the alveolar bone locally in a pinpoint manner.

In a case where said means for causing bone resorption to the alveolar bone is a laser beam applied to said alveolar bone, desirable inflammatory stimuli are given to the alveolar bone, thereby osteoclasts being produced and activated so as to cause bone resorption to the alveolar bone. The laser beam produces a thermal effect serving as an inflammatory stimulus and a mechanical effect serving as an orthodontic force. By focusing the laser beam, it is possible to give effect to a desired portion of the alveolar bone locally in a pinpoint manner.

In a case where said means for causing bone resorption to the alveolar bone is a substance involved in bone resorption, said substance being injected into a surface of said alveolar bone, desirable inflammatory stimuli are given to the alveolar bone, thereby osteoclasts being produced and activated so as to cause bone resorption to the alveolar bone.

In a case where said substance involved in bone resorption is Th17, desirable inflammatory stimuli are given to the alveolar bone, thereby osteoclasts being produced and activated so as to cause bone resorption to said alveolar bone.

### BRIEF DESCRIPTION OF THE SEVERAL VIEWS OF THE DRAWINGS

FIG. 1 is a sectional view of a tooth and surrounding tissue thereof showing an example of an orthodontic method according to the present invention.

FIG. 2 is a sectional view for reference showing a tooth and surrounding tissue thereof

### DETAILED DESCRIPTION OF THE INVENTION

The present invention will now be described in detail with reference to the attached drawings.

The orthodontic method according to the present invention comprises the following steps for example:

(1) The skull of the patient is fixed at three points and images of the oral cavity are taken by means of computed tomography to analyze, on a basis of the images, the arrangement of the teeth, the form of the teeth, the size of the teeth, the form and size of the jawbone, the condition of the periodontal membrane including the existence of bony adhesions, and the condition of the alveolar bone.

(2) The arrangement of the teeth having improved occlusion is three-dimensionally simulated by means of a computer on a basis of the images taken by means of computed tomography.

(3) When the patient is in a growth period, the growth of the jawbone is predicted by means of a computer.

(4) The data obtained first by means of computed tomography is superimposed on the data obtained by simulating the arrangement of the teeth having improved occlusion by means of computed tomography.

(5) The direction and distance of movement of each tooth is three-dimensionally predicted by means of a computer.

(6) Particularly, the changes of the arrangement and position of the tooth root are analyzed. On a basis of the form of the tooth and the positional change of the center of the tooth root, the locus of movement is analyzed by means of a computer. Thereby, the optimum portion for resorption of the alveolar bone is obtained by means of a computer.

(7) Since the skull of the patient is fixed at three points, the optimum portion shown three-dimensionally by a computer can be reproduced.

The orthodontic method according to the present invention further comprises means for causing bone resorption to the alveolar bone and means for applying an orthodontic force to the tooth.

Said means for applying an orthodontic force to the tooth is preferably sonic wave vibration applied to the tooth. Said sonic wave vibration includes ultrasonic wave vibration. Said sonic wave vibration is applied one time or a plurality of times in a pinpoint manner to the tooth and serves as an orthodontic force to the tooth. The frequency of the sonic wave vibration, the length of time of the sonic wave vibration and the power of the sonic wave vibration are such that the sonic wave vibration gives an orthodontic force necessary to move the tooth, and are not limited as far as the sonic wave vibration is not strong to such an extent that it damages the tooth, gingival, periodontal membrane or alveolar bone. The orthodontic force is preferably 200 g to 400 g, for example, according to the tooth. The portion of the tooth to which the orthodontic force is applied is the center of the tooth root in many cases, but varies according as how the tooth is moved. The magnitude of the orthodontic force applied to the tooth, the portion of the tooth to which the orthodontic force is applied, and the direction in which the orthodontic force is applied are calculated by means of computed tomography. The appliance for generating the sonic wave vibration may be an ultrasonic therapy device generally used in dental therapy.

Said means for causing bone resorption to the alveolar bone is preferably sonic wave vibration applied to said alveolar bone. The sonic wave vibration gives a thermal effect. The sonic wave vibration is applied one time or a plurality of times to the alveolar bone to give a thermal stimulation to the alveolar bone, thereby inflammatory stimuli being given the alveolar bone, osteoclasts on a surface of the alveolar bone being activated so as to cause bone resorption to the alveolar bone.

Said means for causing bone resorption to the alveolar bone is preferably a laser beam applied to the alveolar bone. The laser beam gives a thermal effect. The laser beam is applied one time or a plurality of times to the alveolar bone to give a thermal stimulation to said alveolar bone, thereby inflammatory stimuli being given said alveolar bone, osteoclasts on a surface of said alveolar bone being activated so as to cause bone resorption to said alveolar bone.

The laser beam has been generally used in dental therapy because the laser beam suppresses bleeding, and gives less discomfort and pain. However, the use of the laser beam in dental therapy has been limited to the treatment of dental caries, the removal of tartar, the removal of pigmentation, the incision and removal of gums, the treatment of stomatitis, the removal of zonule, etc. The laser beam has not been used in orthodontic treatment. In the present invention, the laser beam is used in the orthodontic treatment.

The intensity of the laser light, the required dose of the laser light, the target of emission, the distance of emission and the time of emission are not limited as far as the thermal stimulation at the portion to which the laser beam is emitted gives inflammatory stimuli and activates osteoclasts. However, the laser beam must not be strong enough to damage the tooth, gingival, periodontal membrane, alveolar bone, etc. In the present invention, any of the following lasers generally used in dental therapy may be used: an argon laser having a wavelength of about 488 nm, a helium neon laser having a wavelength of about 632.8 nm, a semiconductor laser having a wavelength of about 655 nm to 2,000 nm, a Nd-YAG laser having a wavelength of about 1,064 nm, an Er.Cr.YSGG laser having a wavelength of about 2,780 nm, an erbium laser having a wavelength of about 2,940 nm, a carbon dioxide laser having a wavelength of about 10,600 nm, etc. The laser beam is emitted to the target in a pinpoint manner of about 2 to 5 mm, for example.

Said means for causing bone resorption to the alveolar bone may be a substance involved in bone resorption, said substance being injected into a surface of said alveolar bone.

Said substance may be in a liquid or gel state. Said substance may be dissolved in any injectable solution pharmaceutically allowed. Said substance may be injected by any injection means having a container containing said substance and a needle through which said substance is injected.

The place and quantity of injection of said substance are calculated by means of computed tomography.

Said substance may be anything as far as the substance is involved in bone resorption and injectable into the human body. Said substance may preferably be Th17 (T helper 17 cell).

It is already known that said Yh17 introduces quick and strong bone destruction. By injecting TH17 into a surface of the alveolar bone one time or a plurality of times, it is possible to activate the osteoclasts of the alveolar bone so as to cause bone resorption to the alveolar bone.

An example of tooth movements in the orthodontic method according to the present invention will be described below in detail.

(1) First, a portion of the alveolar bone to which bone resorption is to be caused is accurately decided.

(2) Inflammatory stimuli are given to said portion of the alveolar bone to activate the osteoclasts of the alveolar bone and cause bone resorption to said alveolar bone.

(3) The activation of the osteoclasts is maintained by applying a mechanical orthodontic force to the tooth.

(4) Means for causing bone resorption to the alveolar bone by giving inflammatory stimuli to said alveolar bone may be any of the following: a substance involved in bone resorption being directly injected into a surface of the alveolar bone; a thermal stimulation being given to the alveolar bone by a thermal effect of a sonic wave vibration, thereby inflammatory stimuli being given the alveolar bone; or the thermal effect of the laser beam being utilized.

(5) Usually, it is best to apply the orthodontic force in the direction of movement of the tooth. However, it is not always necessary to apply the orthodontic force in the direction of movement of the tooth if the bone resorption portion of the alveolar bone is in proper position.

(6) Even when bone resorption is caused by inflammatory stimuli, the tooth will be reproduced without moving if the tooth does not receive the orthodontic force.

(7) To move the tooth, continuous activation of osteoclasts is necessary. The sonic wave vibration and laser beam must apply some force to push the tooth continuously.

(8) To apply a force to the tooth crown, a supersonic wave toothbrush may be used.

(9) Regarding the sonic wave vibration and the laser beam, it is necessary to clearly discriminate between thermal stimulation and mechanical force (force of pushing the tooth).

(10) The sonic wave vibration may be applied in any of the following directions on a case-by-case basis: upward direction, downward direction, direction toward the cheek, direction toward the tongue, mesial direction, and centrifugal direction.

The present invention will now be described in detail with reference to the attached drawings.

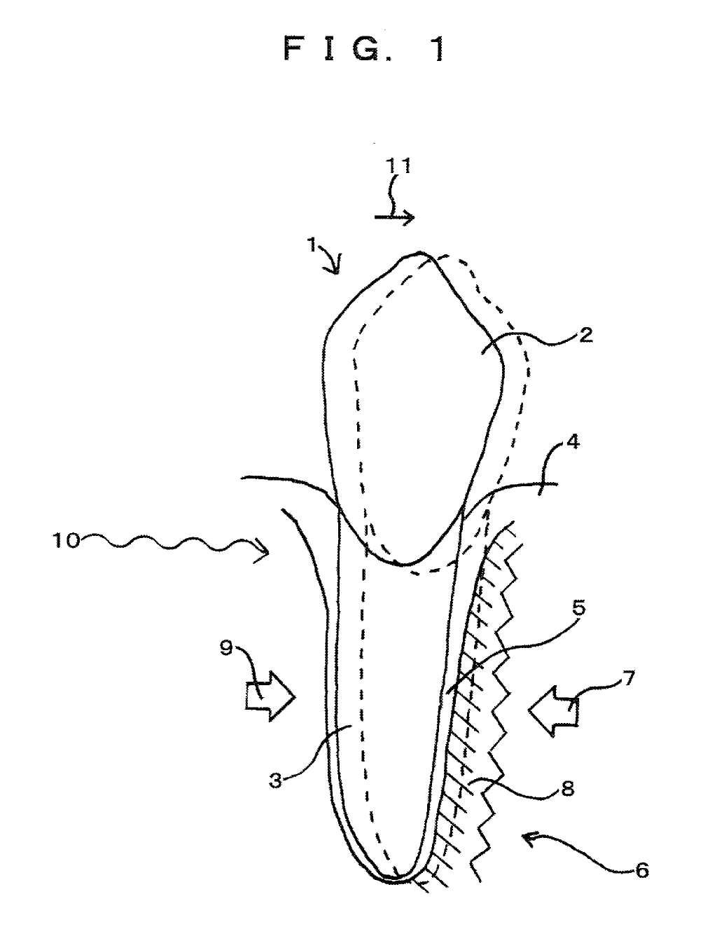

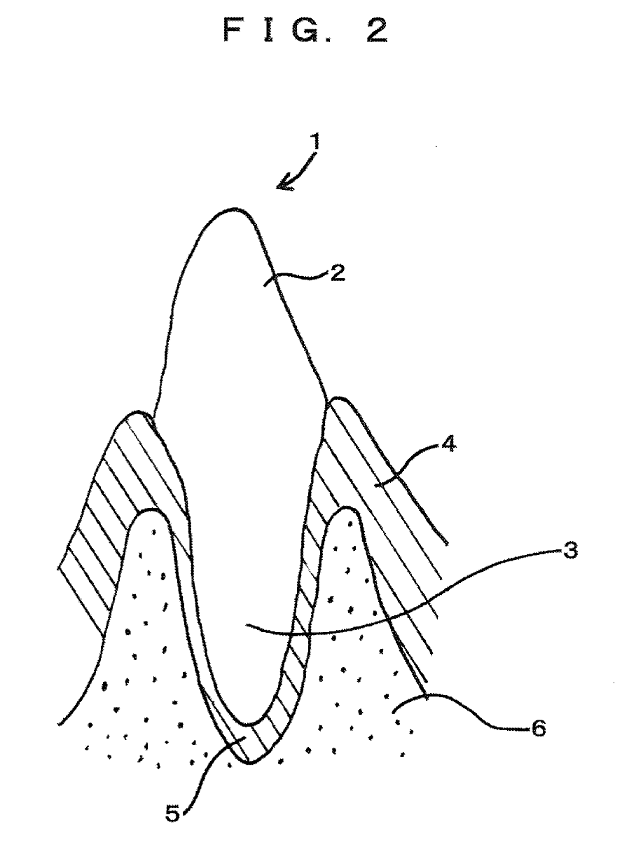

FIG. 1 is a sectional view of a tooth and surrounding tissue thereof showing an example of an orthodontic method according to the present invention. FIG. 2 is a sectional view for reference showing a tooth and surrounding tissue thereof. Numeral 1 represents a tooth. Numeral 2 represents a tooth crown. Numeral 3 represents a tooth root. Numeral 4 represents a gingival. Numeral 5 represents a periodontal membrane. Numeral 6 represents an alveolar bone.

In FIG. 1, bone resorption 8 occurs on a resorption side 7 of the alveolar bone 6, said resorption side 7 being on one side of the tooth 1. An orthodontic force works on an orthodontic side 9 which is on another side of the tooth 1. In the example shown in FIG. 1, means for applying an orthodontic force to the tooth 1 is sonic wave vibration 10. As a result, the tooth 1 moves to a position of a dotted line as shown by an arrow 11.

The sonic wave vibration 10 is not always applied to a central portion of the tooth root 3. The sonic wave vibration 10 may be applied to a portion, other than said central portion, of the tooth root 3 or to the tooth crown 2. The orthodontic force may be applied in different directions.

The sonic wave vibration 10 is applied to the tooth root 3 by, for example, bringing a tip of a sonic wave vibration generator into contact with a surface of the gingival 4.

A thermal effect of the sonic wave vibration 10 or a laser beam gives inflammatory stimuli to the alveolar bone 6, thereby osteoclasts of the alveolar bone 6 being activated, and bone resorption 8 occurs on the alveolar bone 6. It is also possible to cause bone resorption to the alveolar bone 6 by injecting a substance involved in bone resorption into a surface of said alveolar bone 6. Said sonic wave vibration, laser beam and substance involved in bone resorption maybe applied from the tongue side, from the cheek side or from the palate side.

Since the alveolar bone 6 exists inside the gingival 4, said sonic wave vibration, laser beam and substance involved in bone resorption are applied through the gingival 4.

As many apparently widely different embodiments of the present invention may be made without departing from the spirit and scope thereof, it is to be understood that the invention is not limited to the specific embodiments thereof except as defined in the appended claims.

1. An orthodontic method by taking images of the oral cavity in advance by means of computed tomography and analyzing said images,
said orthodontic method comprising means for causing bone resorption to the alveolar bone and means for applying an orthodontic force to the tooth without using an orthodontic appliance.

2. An orthodontic method as claimed in claim 1, wherein said means for applying an orthodontic force to the tooth is sonic wave vibration applied to said tooth.

3. An orthodontic method as claimed in claim 1, wherein said means for causing bone resorption to the alveolar bone is sonic wave vibration applied to said alveolar bone.

4. An orthodontic method as claimed in claim 1, wherein said means for causing bone resorption to the alveolar bone is a laser beam applied to said alveolar bone.

5. An orthodontic method as claimed in claim 1, wherein said means for causing bone resorption to the alveolar bone is a substance involved in bone resorption, said substance being injected into a surface of said alveolar bone.

6. An orthodontic method as claimed in claim 5 wherein said substance involved in bone resorption is Th17.

7. An orthodontic method as claimed in claim 1, wherein said images are taken and analyzed by the following steps:
(1) the skull of the patient being fixed at three points and images of the oral cavity being taken by means of computed tomography to analyze, on a basis of the images, the arrangement of the teeth, the form of the teeth, the size of the teeth, the form and size of the jawbone, the condition of the periodontal membrane including the existence of bony adhesions, and the condition of the alveolar bone,
(2) the arrangement of the teeth having improved occlusion being three-dimensionally simulated by means of a computer on a basis of the images taken by means of computed tomography,
(3) when the patient is in a growth period, the growth of the jawbone being predicted by means of a computer,
(4) the data obtained first by means of computed tomography being superimposed on the data obtained by simulating the arrangement of the teeth having improved occlusion by means of computed tomography,
(5) the direction and distance of movement of each tooth being three-dimensionally predicted by means of a computer, and
(6) the changes of the arrangement and position of the tooth root being analyzed; on a basis of the form of the tooth and the positional change of the center of the tooth root, the locus of movement being analyzed by means of a computer, thereby, the optimum portion for resorption of the alveolar bone being obtained by means of a computer.
